(19) United States
(12) Patent Application Publication
(10) Pub. No.: US 2013/0142763 A1
(43) Pub. Date: Jun. 6, 2013

(54) CROSSLINKED CELLULOSIC POLYMERS

(71) Applicants: William B. Carlson, Seattle, WA (US); Gregory D. Phelan, Cortland, NY (US); Philip A. Sullivan, Seattle, WA (US)

(72) Inventors: William B. Carlson, Seattle, WA (US); Gregory D. Phelan, Cortland, NY (US); Philip A. Sullivan, Seattle, WA (US)

(73) Assignee: EMPIRE TECHNOLOGY DEVELOPMENT LLC, Wilmington, DE (US)

(21) Appl. No.: 13/702,256

(22) PCT Filed: Oct. 27, 2010
(86) PCT No.: PCT/US10/54292

§ 371 Date: Dec. 5, 2012

(51) Int. Cl. A61L 27/20; A61L 27/26

(52) U.S. Cl. CPC A61L 27/20; A61L 27/26
USPC 424/93.7; 514/781; 514/772.1; 514/772.2

(57) ABSTRACT

Crosslinked cellulosic polymers, crosslinked cellulosic polymer hydro-gels, and methods for their synthesis and use are described. The crosslinked cellulosic polymers include one or more cellulosic polymers and a one or more crosslinkers that crosslinks the one or more cellulosic polymers together. The crosslinking can be facilitated with a crosslinking agent capable of linking with a monomer the cellulosic polymer and crosslinking the cellulosic polymer intermoleculerly and/or intramolecularly. Crosslinked cellulosic polymers are well adapted for use in cell and tissue growth in vivo and in vitro. The crosslinked cellulose polymers may also be used as wound care devices.

### BACKGROUND

Many cell growth materials can harm cells in culture and, in the case of bio-implants, they can cause an inflammatory response in the body as the material breaks down. For example, materials such as poly(hydroxylethyl methacrylate), poly(lactic acid), poly(galactic acid), and polyethylene glycols have been investigated as cell growth scaffolds. These materials have desirable mechanical properties, yet they can harm cells in culture and/or induce inflammatory responses in the body as they degrade due to the acidity and/or toxicity of degradation by-products. The inflammatory response can cause swelling, irritation, toxic response, and, in extreme cases, lead to tumor growth.

### SUMMARY

In one aspect, the present disclosure provides a crosslinked cellulosic polymer. In one embodiment, the crosslinked cellulosic polymer can include: one or more cellulosic polymers; and one or more crosslinkers crosslinking the one or more cellulose polymers intermolecularly and/or intramolecularly to form a crosslinked cellulosic polymer. The crosslinked cellulosic polymer may include various moieties, such as a dye, linked to the one or more cellulosic polymers. Generally, the cellulosic polymer can have a molecular weight in a range from about 2000 daltons to about 500,000 daltons. The cellulosic polymer can be selected from the group consisting of hydroxyethylcellulose (HEC) hydroxypropyl cellulose (HPC), carboxymethylcellulose (CMC), hydroxypropyl methylcellulose (HPMC), poly(ethylene glycol) grafted cellulose, and combinations thereof. The crosslinker is the product of a reaction between the one or more cellulosic polymers and a crosslinking agent selected from the group consisting of a dithio diacid, a dicarboxylic acid, an acrylic moiety, a diazide, a styrene, a vinyl carboxylic acid, a urethane, a vinyl acetate, a vinyl ether, a Diels-Alder reagent, disulfides, photopolymerizable moiety, derivatives thereof, and combinations thereof, wherein the crosslinker links to two non-adjacent cellulosic monomers.

In another aspect, the present disclosure provides a composition including a crosslinked cellulosic polymer. In one embodiment, the crosslinked cellulosic polymer can include: one or more cellulosic polymers; and one or more crosslinkers crosslinking the one or more cellulose polymers intermolecularly and/or intramolecularly.

In another aspect, the present disclosure provides a hydrogel including a crosslinked cellulosic polymer. In one embodiment, the crosslinked cellulosic polymer can include one or more cellulosic polymers; one or more crosslinkers crosslinking the one or more cellulose polymers intermolecularly and/or intramolecularly to form a crosslinked cellulosic polymer. In one embodiment, the hydrogel may further include an aqueous medium hydrating the crosslinked cellulosic polymer. The cellulosic polymer may be at least partially water soluble. The hydrogel can include about 0.5 wt % to about 50 wt % of the cellulosic polymer, with up to the balance being an aqueous medium. The aqueous medium can include a buffering agent. Also, the aqueous medium can include at least one cell growth factor. The hydrogel can be porous or non-porous. In one embodiment, the hydrogel may include pores of sufficient dimension for culturing one or more cells within the pores.

In another aspect, the present disclosure provides a cell growth scaffold including a crosslinked cellulosic polymer. In one embodiment, the cell growth scaffold may include one or more cells on or in the scaffold. The cells can be alive or dead, as well as only one cell type or more than one cell type. In another embodiment, the cell growth scaffold may include indicators such as dyes. In another aspect, the cell growth scaffold may include drugs from either natural or synthetic sources. For example witch hazel, theobromines, acetaminophen, acetasalysilic acid, or any number of anti-inflammatory or anti-cancer compounds can be included in the cell growth scaffold. In another aspect, the cell growth scaffold may include an anesthetic. In another aspect, the cell growth scaffold may include a cell growth factor.

The cell growth scaffold may be porous or not porous. In one embodiment, the cell growth scaffold is porous. The cell growth scaffold can include one or more pores configured as any one of the follows: the pores have a dimension sufficient for cell growth therein; the pores have a dimension that corresponds with a porogen; the pores are formed by removal of a porogen from crosslinked cellulosic polymers; the pores have a dimension larger than about 50 nm; the pores have a dimension from about 50 nm to about 900 nm; the pores have a dimension from about 1 micron to about 10 microns; the pores have a dimension sufficient for culturing a bacteria; the pores have a dimension larger than about 10 micron; the pores have a dimension from about 10 microns to about 100 microns; the pores have a dimension sufficient for culturing a prokaryotic cell or a eukaryotic cell; the pores are smaller than 1 micron; or the pores are smaller than a bacteria, and can filter bacteria. The pore size is dependent upon the bead size or otherwise method used to create the porogen. Beads useful as porogens can range from the nanometer size on up. The required pore size is dependent upon the application that the pore is being used for.

In one aspect, the present disclosure provides an endoprosthesis including a crosslinked cellulosic polymer.

In one aspect, the present disclosure provides a tissue scaffold including a crosslinked cellulosic polymer.

In one aspect, the present disclosure provides a tissue implant article including a crosslinked cellulosic polymer

In one aspect, the present disclosure provides a cell culture insert including a crosslinked cellulosic polymer.

In one aspect, the present disclosure provides a wound dressing including a crosslinked cellulosic polymer.

In one aspect, a method of growing cells can include: providing the hydrogel prepared from the crosslinked cellulosic polymer; and growing one or more cells on the hydrogel.

In one aspect, a method for crosslinking a cellulosic polymer can include: providing one or more cellulosic polymers; providing one or more crosslinking agents; and crosslinking the one or more cellulosic polymers with the one or more crosslinking agents so as to form a crosslinked cellulosic polymer. The cellulosic polymers can be a cellulose derivative selected from the group consisting of hydroxyethylcellulose (HEC) hydroxypropyl cellulose (HPC), carboxymethylcellulose (CMC), hydroxypropyl methylcellulose (HPMC), poly(ethylene glycol) grafted cellulose, and combinations thereof. The crosslinking agents can be selected from the group of a dithio diacid, a dicarboxylic acid, an acrylic moiety, a diazide, a styrene, a vinyl carboxylic acid, a urethane, a vinyl acetate, a vinyl ether, a Diels-Alder reagent, disulfides, photopolymerizable moiety, derivatives thereof, and combinations thereof.

In one aspect, a method for crosslinking a cellulosic polymer can include: providing a cellulosic polymer in a reaction medium (e.g., a liquid reaction medium such as an aqueous solution), wherein the cellulosic polymer includes reactable functional groups; activating at least a subset of the reactable functional groups on the cellulosic polymer with a coupling reagent to form a reactive intermediate; and reacting a crosslinking agent with the reactive intermediate to form either a crosslinked or a crosslinkable cellulosic polymer. The method can also include treating the crosslinkable cellulosic polymer to convert the crosslinkable cellulosic polymer to a crosslinked cellulosic polymer. For example, the treating can form a disulfide bond, for example, through an oxidizing agent, such as hydrogen peroxide.

In one aspect, a crosslinked cellulosic polymer can be uncrosslinked. In some instances, the crosslinker of a crosslinked cellulosic polymer can be reversible so that when the crosslinked cellulosic polymer is contacted by a crosslink reversing agent the crosslinker degrades and unlinks the cellulosic polymer. When the cellulosic polymer is crosslinked, treating the crosslinked cellulosic polymer with the crosslink reversing agent at least partially reverses the crosslinking to form a crosslinkable cellulosic polymer. For example, the crosslinker can include a disulfide bond, which can be treated with a reducing agent such as dithiothreitol. Thus, dithiothreitol can be a crosslink reversing agent for disulfide crosslinkers.

In one embodiment, the crosslinking method can result in about 0.01% to about 20% of the functional groups being linked to a crosslinking agent.

In one embodiment, the crosslinking method can include the cellulosic polymer being provided with reactable functional groups. In one embodiment, the crosslinking agent is provided with reactable functional groups. Examples of the reactable functional groups include members selected from the group consisting of a carboxylic acid, aldehyde, hydroxyl, amine, thiol, or combination thereof. Alternatively, the crosslinking method can include reacting the cellulosic polymer with a coupling reagent so as to activate the cellulosic polymer with reactable functional groups. In another embodiment, the crosslinking method can include reacting the crosslinking agent with a coupling reagent so as to activate the crosslinking agent and facilitate the reaction between the crosslinking agent and the reactable functional groups. The coupling reagent can include a member selected from the group of hydroxybenzotriazole (HOBt), N,N′-dicyclohexylcarbodiimide (DCC), N,N′-diisopropylcarbodiimide (DIC), 1-Ethyl-3-(3-dimethylaminopropyl)carbodiimide (EDC), and combinations thereof.

In one embodiment, the crosslinking method can include: adding at least two coupling agents to the cellulosic polymer in aqueous solution to form a cellulosic polymer activated ester capable of reacting with the crosslinking agent to form a bond between the crosslinking agent and the cellulosic polymer. In one embodiment, the at least two coupling agent may include hydroxybenzotriazole (HOBt) and a carbodiimide reagent.

In one embodiment, a method of wound healing can include: introducing a wound dressing into a wound of a subject, the wound dressing including a crosslinked cellulosic polymer.

In one embodiment, a method for culturing cells can include: introducing a cell culture article of manufacture into a cell culture chamber, where the cell culture article of manufacture includes a crosslinked cellulosic polymer; and culturing one or more cells in the cell culture chamber with the cell culture article of manufacture such that the one or more cells migrate and/or proliferate on or within the cell culture article.

In one embodiment, a method of implanting cells in a subject can include: implanting a tissue scaffold containing one or more cells into a subject, the tissue scaffold including a hydrogel having a crosslinked cellulosic polymer.

The foregoing summary is illustrative only and is not intended to be in any way limiting. In addition to the illustrative aspects, embodiments, and features described above, further aspects, embodiments, and features will become apparent by reference to the following detailed description.

### BRIEF DESCRIPTION OF THE DRAWINGS

To further clarify the above and other advantages and features of the present disclosure, a more particular description of the disclosure will be rendered by reference to specific embodiments thereof which are illustrated in the appended drawings. It is appreciated that these drawings depict only illustrated embodiments of the disclosure and are therefore not to be considered limiting of its scope. The disclosure will be described and explained with additional specificity and detail through the use of the accompanying drawings in which:

FIG. 1 provides a schematic of an illustrative embodiment of a cellulosic polymer that is crosslinked to another cellulosic polymer.

FIG. 2 provides a schematic of an illustrative embodiment for crosslinking a crosslinkable cellulosic polymer to another crosslinkable cellulosic polymer.

FIG. 3 provides a schematic of an illustrative embodiment for crosslinking a cellulosic polymer with a disulfide containing crosslinker.

FIG. 4 provides a schematic of an illustrative embodiment showing reversible crosslinking with a disulfide containing crosslinker.

FIG. 5 provides a schematic of an illustrative embodiment of a cellulosic polymer that is substituted with a crosslinkable acrylic group.

FIG. 6 provides a schematic of an illustrative embodiment for crosslinking the cellulosic polymer of FIG. 5.

FIGS. 7A-7D provide embodiments of Diels Alder dienophile and diene and corresponding reactions with the cellulosic polymer (HEC) as well as crosslinking reaction between the dienophile and diene to crosslink the HEC polymers (Sullivan, P. A.; Olbricht, B. C.; Akelaitis, A. J. P.; Mistry, A. A.; Liao, Y.; Dalton, L. R., Tri-component Diels-Alder Polymerized Dendrimer Glass Exhibiting Large, Thermally Stable, Electro-optic Activity. J. Mater. Chem. 2007, DOI: 10.1039/b701815k).

### DETAILED DESCRIPTION

### I. Introduction

In the following detailed description, reference is made to the accompanying Figures, which form a part hereof. In the Figures, similar symbols typically identify similar components, unless context dictates otherwise. The illustrative embodiments described in the detailed description, Figures, and claims are not meant to be limiting. Other embodiments may be utilized, and other changes may be made, without departing from the spirit or scope of the subject matter presented here.

The present disclosure relates inter alia to crosslinked cellulosic polymers, crosslinked cellulosic polymer compositions, articles of manufacture (e.g., cell culture scaffolds or inserts) prepared from crosslinked cellulosic polymers and methods for their synthesis and use. The articles of manufacture having the crosslinked cellulosic polymers can be used as substrates for cells in vitro and in vivo. The crosslinked cellulosic polymers disclosed herein possess excellent mechanical properties that make them well-suited for use as a scaffold for tissue growth for bio-medical implant (e.g., endoprosthesis) applications. Moreover, the crosslinked cellulosic polymers disclosed herein are non-toxic in and of themselves, and their breakdown products (i.e., oligosaccharides and sugar monomer units) are compatible both to cells in culture and to tissues in and surrounding an endoprosthesis formed from the crosslinked cellulosic polymers.

In one embodiment, the present disclosure provides a composition including a crosslinked cellulosic polymer. The crosslinked cellulosic polymer may include a cellulosic polymer that is crosslinked with a crosslinker. The crosslinker can be formed from reacting the cellulosic polymer with a crosslinking agent and therefore crosslinking the cellulosic polymer intermolecularly and/or intramolecularly. The cellulosic polymer may include one or more hexose monomer units and/or a polyhexose. In one embodiment, the cellulosic polymer may include one or more pentose monomer units and/or a polypentose. In one embodiment, the present disclosure provides a hydrogel including a crosslinked cellulosic polymer. Such a crosslinked cellulosic polymer-based hydrogel can include an aqueous medium and a crosslinked cellulosic polymer.

In one embodiment, a method for crosslinking a cellulosic polymer can include crosslinking one or more cellulosic polymers with one or more crosslinking agents to provide a crosslinked cellulosic polymer. The reaction between the crosslinking agent and one or more cellulosic monomers forms a crosslinking agent reaction product (e.g., crosslinker) that crosslinks two or more cellulosic monomers. The method can also include functionalizing one or more cellulosic monomers of the cellulosic polymer(s) with reactive groups that can react with the crosslinking agent in order to crosslink the cellulosic polymer(s).

A method for making a crosslinked cellulosic polymer can include providing a cellulosic polymer in a reaction medium (e.g., an aqueous solution), wherein the cellulosic polymer includes reactable functional groups, activating at least a subset of the functional groups on the cellulosic polymer with a coupling agent to form a reactive intermediate, and reacting a crosslinking agent with the reactive intermediate to form either a crosslinked or a crosslinkable cellulosic polymer. The crosslinkable polymer is then crosslinked intramolecularly or intermolecularly.

As used herein, the term “cellulose” refers to an organic compound with the formula (C₆H₁₀O₅)ₙ, which is a polysaccharide having a linear chain of β(1→4) linked D-glucose units and having the structure of Formula 1, where n is any integer.

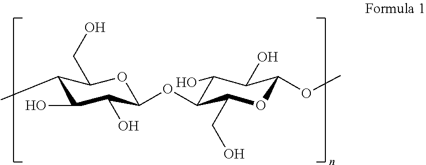

As used herein, the term “cellulose derivative” refers to cellulosic polymers that are based on cellulose and derivatized with functional groups that are generally not found in naturally occurring celluloses. Such functional groups are selected to serve a variety of purposes or functions including, but not limited to: reactive groups to increase reactivity with a crosslinking agent to form crosslinkers; alkoxy groups to increase solubility; providing a substituent extended from the ring for reaction with reactive groups and/or a crosslinking agent; polymers to provide functionalities associated with the graft polymers, such as polyethylene glycol (PEG) to increase solubility; colorometric, fluorometric, or other optically visible functional groups to provide optical detection; or others.

Examples of cellulose derivative include, but are not limited to, hydroxyalkylcelluloses (HAC), hydroxyethylcellulose (HEC), hydroxypropyl cellulose (HPC), carboxymethylcellulose (CMC), hydroxypropyl methylcellulose (HPMC), poly(ethylene glycol) grafted cellulose, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, and combinations thereof.

An example of cellulose derivative, hydroxyalkylcellulose, is shown in Formula 2 below. In Formula 2, R can be hydrogen or any alkoxy group with a free hydroxyl group on the end. Also, the structure in Formula 2 can be a general formula for a cellulosic polymer that includes a cellulose derivative with each R being independently selected from substituents selected from the group of hydrogen, C₁-C₂₄ alkyl, C₂-C₂₄ alkenyl, C₂-C₂₄ alkyllyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano(—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—S₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O—)), phospho (—PO₂), phosphino (—PH₂)₅ derivatives thereof, and combinations thereof

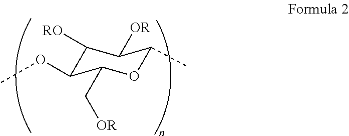

As used herein, the term “cellulosic polymer” refers to a polymer that is either a cellulose or a cellulose derivative.

As used herein, the term “crosslinking agent” refers to one or more molecules that react with the cellulosic polymer in order to crosslink a monomer of the cellulosic polymer with another monomer either intramolecularly or intermolecularly. Often, a crosslinking agent can include a molecular construct that can react at two or more ends of the molecule with the monomer of the cellulosic polymer. Also, the crosslinking agent can include a molecular construct that can react with functionalized groups or substituents of a derivatized cellulosic polymer. A crosslinking agent reacts with cellulosic polymer so as to form the crosslinker that crosslinks the cellulosic polymer intermolecularly or intramolecularly. As such, the crosslinking agent forms the crosslinker. Thus, a “crosslinker” is a reaction product obtained from reacting one or more cellulosic monomers of a cellulosic polymer with a crosslinking agent. The crosslinking agents are described in more detail herein.

As used herein, the term “hydrogel” refers to an aqueous network of crosslinked cellulosic polymers. Hydrogels, which are highly absorbent, can contain over 99% water. Hydrogels also possess a degree of flexibility very similar to natural tissue, due to their significant water content. Because of their properties, hydrogels are currently used as scaffolds for cell/tissue growth in tissue engineering and tissue repair. As such, hydrogels usually include a porous network that allows cells to grow, propagate, and expand throughout the hydrogel.

As used herein, the term “crosslinked” refers to a cellulosic polymer in which cellulosic polymer molecules are coupled to crosslinkers that link the cellulosic polymer monomers either intermolecularly or intramolecularly.

As used herein, the terms “crosslinkable” refers to a cellulosic polymer in which cellulosic polymer molecules are linked to coupling agents that are capable of coupling together for crosslinking cellulosic polymer molecules either intermolecularly or intramolecularly. That is, coupling agents are bound to the cellulosic polymer molecules, but, in the “crosslinkable” state, the coupling agents are not bound to each other or to more than one monomer. Once reacted together, the coupling agents form the linker that crosslinks the cellulosic polymer. An example can include coupling agents having thiol groups that can react to form a disulfide crosslinker. One will also appreciate that some crosslinkable coupling agents permit reversible crosslinking, such as crosslinkers that include disulfide groups that can be broken into separate thiol groups. For example, crosslinking agents that are capable of forming disulfide linkages are both crosslinkable and reversible. Coupling agents that include thiols may also be considered to be crosslinking agents as they can form crosslinkers having disulfides.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred substituents identified as “C₁-C₆ alkyl” or “lower alkyl” contains 1 to 3 carbon atoms, and particularly preferred such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The terms “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, preferably 2 to 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, preferably 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred substituents identified as “C₁-C₆ alkoxy” or “lower alkoxy” herein contain 1 to 3 carbon atoms, and particularly preferred such substituents contain 1 or 2 carbon atoms (i.e., methoxy and ethoxy).

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 20 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 20 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred aralkyl groups contain 6 to 24 carbon atoms, and particularly preferred aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, and fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

The term “hydrocarbyl” refers to univalent hydrocarbyl radicals containing 1 to about 30 carbon atoms, preferably 1 to about 24 carbon atoms, more preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, heteroatom-containing alkenyl, and heteroatom-containing aryl.”

### II. Cellulosic Polymer Compositions

In one embodiment, a composition having a crosslinked cellulosic polymer is disclosed. The composition is useful as a substrate for growing cells, and can, for example, be adapted for use as a cell growth scaffold for an endoprosthesis. In one aspect, the crosslinked cellulosic polymer can include one or more cellulosic polymers and one or more crosslinkers that crosslink the cellulosic polymer either intermolecularly or intramolecularly. For example, the cellulosic polymers disclosed herein include a number of hydroxyl groups that can be reacted with the various crosslinking agents disclosed herein to form the crosslinkers and therefore provide crosslinked cellulosic polymer.

The crosslinking agent can include one or more molecules that are capable of forming at least a first bonding interaction with functional groups found on a monomer of a cellulosic polymer, and forming at least a second bonding interaction with functional groups found in either another monomer of a cellulosic polymer molecule (i.e., intermolecular crosslinking) or a monomer within the same cellulosic polymer molecule (i.e., intramolecular crosslinking).

In one aspect, the cellulosic polymer is a cellulose derivative. Naturally occurring cellulose is a polymer consisting of D-glucose monomer units held together by alternating β-1,4-glycosidic bonds. Under strongly alkali conditions the various hydroxyl moieties on cellulose can be substituted with various moieties such as but not limited to ethoxy, propoxy and other useful functional entities that provide substitution possibilities as well as increased solubility. In one aspect, possible substituents may include, but are not limited to, straight or branched substituted or unsubstituted C₁-C₂₀ alkane, straight or branched substituted or unsubstituted C₁-C₂₀ alkene, straight or branched substituted or unsubstituted C₁-C₂₀ alkyne, straight or branched substituted or unsubstituted C₁-C₂₀ carboxylic acid, straight or branched substituted or unsubstituted C₁-C₂₀ ester, phenyl, benzyl, halogen, straight or branched substituted or unsubstituted alkoxy, primary amine, secondary amine, tertiary amine, azide, azo, phosphate, phosphine, sulfide, sulfonyl, substituted or unsubstituted cycloalkyl, substituted or unsubstituted aryl, branched or unbranched or cyclic substituted or unsubstituted arylalkyl, or combinations thereof. In another aspect, possible substituents may include, but are not limited to, CH₂CH₂OH, CH₂CH(OH)CH₃, CH₂CO₂H, CH₃, and combinations thereof. Other possible functional groups or substituents are also described above.

Suitable examples of specific cellulose derivative include, but are not limited to, hydroxyethylcellulose (HEC), hydroxypropyl cellulose (HPC), carboxymethylcellulose (CMC), hydroxypropyl methylcellulose (HPMC), poly(ethylene glycol) grafted cellulose, and combinations thereof.

In one aspect, the cellulosic polymer has an average molecular weight of about 2000 daltons (Da), about 5000 Da, about 10,000 Da, about 15,000 Da, about 20,000 Da, about 25,000 Da, about 30,000 Da, about 35,000 Da, about 40,000 Da, about 45,000 Da, about 50,000 Da, about 60,000 Da, about 70,000 Da, about 80,000 Da, about 90,000 Da, about 100,000 Da, about 125,000 Da, about 150,000 Da, about 175,000 Da, about 200,000 Da, about 250,000 Da, about 300,000 Da, about 350,000 Da, about 400,000 Da, about 450,000 Da, about 500,000 Da, or any value therebetween.

High molecular weight cellulosic polymers form highly viscous solutions when the polymer is dissolved in aqueous solution, even at low concentrations. Molecular weight has a logarithmic effect on viscosity; thus, small increases in molecular weight greatly increase viscosity. For example, at a molecular weight of one million daltons, 2% solutions of cellulose can be gelled; even without crosslinking However, such solutions can be very difficult to work with because of their high viscosity. Moreover, an interesting phenomenon commonly observed in carbohydrate polymer solutions is that they exhibit non-Newtonian viscosity. When shear force is applied, for instance, solutions of carbohydrate polymers can become much less viscous. The reason for this behavior is believed to be related to the formation and severing of inter- and/or intra-chain hydrogen bonding. When in the static state the polymer chains form hydrogen bonds between each other. The hydrogen bonding acts to extend the effective chain length of the polymers, increasing viscosity. However, the hydrogen bonds are weaker than covalent bonds and are broken when the solution is placed under shear. Once under shear, the chain length of the polymer is much shorter resulting in a decrease in viscosity.

This tendency to change viscosity under shear can be overcome by crosslinking the cellulosic polymer chains with a crosslinker. The crosslinking agent is capable of forming a crosslinking interaction (e.g., covalent boding cellulosic monomers) either intramolecularly within a cellulosic polymer or intramolecularly between cellulosic polymer molecules. Suitable examples of crosslinking agents include but are not limited dithio diacid, a dicarboxylic acid, an acrylic moiety, a diazide, a styrene, a vinyl carboxylic acid, a urethane, a vinyl acetate, a vinyl ether, a Diels-Alder reagent, disulfides, photopolymerizable moiety, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, derivatives thereof, and combinations thereof.

Suitable examples of dithio diacids include, but are not limited to, dithio dicarboxylic acid, dithio dipropanoic acid, dithio dibutanoic acid, dithio dipentanoic acid, dithio dihexanoic acid, and derivatives and combinations thereof. Specific examples of dithio diacids can include 16-carboxyhexadecyl disulfide, 5,5′dithiobis(2-nitrobenzoic acid), 2,2′-dithiodibenzoic acid, 4,4′-dithiodibutyric acid, 3,3′-dithiodipropionic acid and 6,6′-dithiodinicotinic acid. As the chain length of the dithio diacid, or other crosslinker/crosslinking agent increases more surfactant like behavior forms, which can be beneficial.

In one aspect, the crosslinking is reversible. For example, the crosslinking with the dithio or diacids listed above can be reversed with the addition of dithiothreitol (DTT) or a similar reducing reagent that can break the disulfide linkage in the crosslinker. The cross-linking can be reinitiated by addition of an oxidizing agent such as, but not limited to, hydrogen peroxide.

In one aspect, suitable examples of dicarboxylic acids include, but are not limited to, oxalic acid, malonic acid, succinic acid, glutaric acid, adipic acid, pimelic acid, suberic acid, azelaic acid, sebacic acid, phthalic acid, maleic acid, isophthalic acid, terephthalic acid, and derivatives and combinations thereof.

In one embodiment, the crosslinking agent can include an acrylic moiety such as but not limited to acrylic acid, methacrylic acid, hydroxyethyl methacrylate, hydroxypropyl methacrylate, acrylamide, glucose methacrylate, gallactose methacrylate, aminoethyl methacrylate, derivatives and combinations thereof.

In one embodiment, the crosslinking agent includes styrene, 4-vinylbenzoic acid, 4-vinylbenzenesulfonic acid, vinyl pyridine, vinyl phenol, divinylbenzene, 4-cyanostyrene, or derivative or combination thereof.

In one embodiment, the crosslinking agent includes a vinyl carboxylic acid, vinyl acetate, vinyl alcohol, vinyl amine, vinyl propionate, vinylbutyrate, vinylbutryaldehyde, or derivative or combination thereof.

In one embodiment, the crosslinking agent includes a disulfide such as but not limited to 16-carboxyhexadecyl disulfide, 5,5′dithiobis(2-nitrobenzoic acid), 2,2′-dithiodibenzoic acid, 4,4′-dithiodibutyric acid, 6,6′-dithiodinicotinic acid, 3,3′-dithiodipropionic acid, derivatives thereof and combinations thereof.

In one aspect, the crosslinking agent is photoreactive, thermoreactive, and/or catalytic. In one embodiment, the photoreactive crosslinking agent may contain a photoreactive crosslinkable moiety. In one embodiment, the crosslinking agent may contain a thermoreactive crosslinkable moiety. In one embodiment, the crosslinking agent may contain a catalytic crosslinkable moiety. The representative crosslinkable moieties include, without limitation, an acrylic moiety, styrenic moiety, alkyene moiety, alkyn moiety, diene moiety, dinenothiod moiety, and epoxy moiety,

The cellulosic polymer may include hexose units, pentose units, or a combination thereof. In one aspect, the cellulosic polymer can include one or more hexose units, and/or the cellulosic polymer can be conjugated or grafted to a polyhexose that has monomer units selected from the group consisting of allose, altrose, glucose, mannose, gulose, idose, galactose, talose, psicose, fructose, sorbose, tagatose, and combinations and derivatives thereof. Many naturally occurring polysaccharides are polyhexoses. For example, celluloses, pectins, and amylopectins are glucose polymers. For comparison, chitins and chitosans are polymers composed of N-acetylglucosamine (chitin) and N-acetylglucosamine and glucosamine (chitosan).

In one aspect, the cellulosic polymer can include one or more pentose units, and/or the cellulosic polymer can be conjugated or grafted to a polypentose. Suitable examples of pentose monomer units that may be included in the cellulosic polymer include, but are not limited to, ribose, arabinose, xylose, lyxose, ribulose, xylulose, and combinations and derivatives thereof.

In one aspect, the cellulosic polymer can be conjugated or grafted with a starch, a pectin, an amylopectin, or a derivative thereof. Starch is a polysaccharide carbohydrate consisting of a large number of glucose units joined together by glycosidic bonds.

In one aspect, the composition may include a crosslinking initiator. The crosslinking initiator can be capable of initiating crosslinking intermolecularly and intramolecularly through, for example, radical reaction, carbanion reaction, carbocation reaction, nucleophilic substitution, and cycloaddition. The initiator may be a photo-initiator, thermo-initiator, or a catalyst. The intiator may be a carbonitrile or a phenone. Representative initiators may include hydrogen peroxide, benzoyl peroxide, persulfate, AIBN, ABCN, nitrile, and benzo phenone. Catalysts, for example ferrous, can be added to catalyze either initiation or crosslinking reactions.

In one aspect, the composition includes a reporter molecule. The reporter molecule may be, but is not limited to, a visible dye, fluorescent dye, an isotope label, a radioactive tag, a molecular label, a drug label, a cleavable label, or a hydrolyzable label. The reporter molecule may be covalently or non-covalently coupled to the cellulosic polymer. Non-limiting examples of visible or fluorescent dye reporter molecules may include fluorescein isothiocyanate (FITC), fluorescein, rhodamine, coumarin, and cyanine as well as others. Non-limiting examples of isotope labels may include ¹⁸O, ¹⁵N, ¹³C, or ²H. Non-limiting radioactive tags may include ¹⁸F, ³H, or ¹⁴C. Non-limiting examples of drug labels may include acetyl salicylic acid, nicotine, ciprosloxacin, quinolone, levosloxacin, provasloxacin, γ-hydroxybutanoic acid, modafinil, ampakine, yohimbine, folinic acid, β-cis-retinoic Acid, tretinoin, citric acid, ascorbic acid, and acetaminophen.

In one embodiment, the reporter molecule may be coupled to the polymer through the same reaction schemes as described with regard to the crosslinking agents and coupling agents being linked to the cellulosic polymers. For example, the hydroxyl groups of the cellulosic polymer can be functionalized as described and coupled to a reporter molecule that also has a reactive functional group.

The cellulosic polymers can also be grafted with various other types of polymers so that the properties of these other polymers are incorporated into the cellulosic polymer as well as the crosslinked cellulosic polymer. As used, “grafting” of cellulosic polymers with other polymers can be performed by covalently linking a polymer to a monomer of a cellulosic polymer. The polymer can be coupled either directly to the monomer by replacing a hydroxyl group or coupled indirectly through a linker. For example, a water soluble polymer, such as polyethylene glycol (PEG), can be grafted to the glucose units of cellulosic polymers to increase water solubility. Also, water insoluble polymers such as polyethylene or polystyrene can be grafted to the cellulosic polymers to reduce water solubility. Any type of polymer can be grafted to the cellulosic polymer to form a hybrid cellulosic polymer having the properties of the polymer. Examples of polymer molecular weights can include about 400 to about 40,000, or even higher, or from 700 to 30,000, or from about 1,000 to about 20,000, or from about 5,000 to about 10,000.

In some instances, the cellulosic polymer can be grafted with non-biodegradable materials (e.g., biostable polymers), which can be biocompatible and useful for various medical devices and drug delivery systems for external use or situations where biodegradability is not necessary such as in extractable medical devices that are removed from a body after use. Some examples of non-biodegradable polymers that can be grafted to a cellulosic polymer can include polyethylenes, polypropylenes, polyvinylchlorides, polystyrenes, and polycarbonates as well as others. Examples of some biodegradable polymers that can be grafted to the cellulosic polymers can include polyhydroxyalkanoates, polyhydroxybutyrate-valerate, polylactic acid, polylactates, polyglycolic acids, polyglycolides, polycaprolactones, polyvinyl alcohols, combinations thereof, and others.

In another embodiment, a hydrogel including the crosslinked cellulosic polymer is disclosed. The hydrogel can be used as a substrate for growing cells, such as, for example, being adapted for use as a cell growth scaffold. In one embodiment, the hydrogel may include an aqueous medium. In one aspect, the hydrogel may include a buffering agent as the aqueous medium. Suitable examples of buffering agents that are commonly used in biology include, but are not limited to, 3-{[tris(hydroxymethyl)methyl]amino}propanesulfonic acid (“TAPS”), N,N-bis(2-hydroxyethyl)glycine (“Bicine”), tris(hydroxymethyl)methylamine (“Tris”), N-tris(hydroxymethyl)methylglycine (“Tricine”), 4-2-hydroxyethyl-1-piperazineethanesulfonic acid (“HEPES”), 2-{[tris(hydroxymethyl)methyl]amino}ethanesulfonic acid (“TES”), 3-(N-morpholino)propanesulfonic acid (“MOPS”), piperazine-N,N′-bis(2-ethanesulfonic acid) (“PIPES”), saline sodium citrate (“SSC”), 2-(N-morpholino)ethanesulfonic acid (“MES”), phosphate buffered saline (“PBS”), and combinations thereof.

In one aspect, the hydrogel may include at least one cell growth factor. The cell growth factor may be coupled to the crosslinked cellulosic polymer covalently or noncovalently. A growth factor can include a substance capable of stimulating cellular growth, proliferation and cellular differentiation, and regulate a variety of cellular processes. For instance, cell growth factors may include, but are not limited to, a carbon source such as glucose needed for cell growth, various salts (e.g., calcium chloride, potassium chloride, magnesium sulfate, sodium chloride, and monosodium phosphate), vitamins (e.g., folic acid, nicotinamide, riboflavin, and B-12), proteins, cytokines, and growth factors such as steroid hormones and proteins. In another aspect, the hydrogel includes the cell growth factor up to about 1 wt %, about 2 wt %, about 3 wt %, about 1 wt %, about 1 wt %, about 1 wt %, about 1 wt %, about 1 wt %, about 1 wt %, about 4 wt %, about 5 wt %, about 6 wt %, about 7 wt %, about 8 wt %, about 9 wt %, about 10 wt % of the hydrogel or any range therebetween.

In one aspect, the hydrogel can include about 0.5 wt % to about 50 wt % crosslinked cellulosic polymer and the balance can include an aqueous medium. In another aspect, the hydrogel includes cellulosic polymer at about 1 wt %, about 2 wt %, about 3 wt %, about 1 wt %, about 1 wt %, about 1 wt %, about 1 wt %, about 1 wt %, about 1 wt %, about 4 wt %, about 5 wt %, about 6 wt %, about 7 wt %, about 8 wt %, about 9 wt %, about 10 wt %, about 15 wt %, about 20 wt %, about 25 wt %, about 30 wt %, about 35 wt %, about 40 wt %, about 45 wt %, or about 40 wt % cellulosic polymer, or any range therebetween, and the balance including an aqueous medium.

One will appreciate, however, that the weight percent of the cellulosic polymer in the hydrogel is at least partially a function of the molecular weight of the cellulosic polymer used. As was discussed in greater detail above, the molecular weight of the cellulosic polymer has a logarithmic effect on the viscosity of the uncrosslinked cellulosic polymer solution. For example, a 50 wt % cellulosic polymer solution may be workable where the cellulosic polymer has a molecular weight of 2000 Da, whereas a only a 1-2 wt % cellulosic polymer solution may be needed with a cellulosic polymer having a molecular weight of 1,000,000 Da or higher.

Another useful metric for determining the weight percent of the cellulosic polymer to be included in the hydrogel can therefore be based on measurements of the viscosity of the uncrosslinked cellulosic polymer solution. Measurements of the viscosity of the uncrosslinked cellulosic polymer solution can be based on the Brookfield method. All Brookfield viscometers employ the principle of rotational viscometry. The viscosity of a product is determined by the amount of torque that is required for a spindle to rotate at a constant speed while immersed in a fluid. This amount of torque is proportional to the viscous drag on the immersed spindle, and thus to the viscosity of the fluid. In one aspect, the uncrosslinked cellulosic polymer solution can have a Brookfield viscosity in a range from about 50 to about 200, about 60 to about 175, or about 75 to about 150.

The amount of crosslinking can also be varied depending on the desirable property of the crosslinked cellulosic polymer. The amount of crosslinking can be regulated by controlling the ratio of crosslinking agent and cellulosic monomer. For example, the crosslinking can be expressed with respect to the percentage of monomers being crosslinked, which can range from about 1% to about 90% by weight, about 10% to about 80%, about 20% to about 70%, about 30% to about 60%, or about 40% to about 50% or any range therebetween. The lower the crosslinking the more porous the hydrogel can be, and vice versa.

In one embodiment, the crosslinked cellulosic polymer composition as dry or hydrogel can be used as a wound dressing.

The crosslinked cellulosic polymer can include one or more pores configured as any one of the follows: the pores have a dimension sufficient for cell growth therein; the pores have a dimension that corresponds with a porogen; the pores are formed by removal of a porogen from crosslinked cellulosic polymer; the pores have a dimension larger than about 50 nm; the pores have a dimension from about 50 nm to about 900 nm; the pores have a dimension from about 1 micron to about 10 microns; the pores have a dimension sufficient for culturing a bacteria; the pores have a dimension larger than about 10 micron; the pores have a dimension from about 10 microns to about 100 microns; the pores have a dimension sufficient for culturing a prokaryotic cell or a eukaryotic cell; the pores are smaller than 1 micron; or the pores are smaller than a bacteria, and can filter bacteria. The pores can be formed by controlled crosslinking as well as by using porogens. As used herein, a “porogen” is a substance such as a particle or pocket of material that forms a pore by removing the porogen from the crosslinked cellulosic polymer.

Pores can be created by the template method. The hydrogel is injected into a template, polymerized, and then the template is removed to create the porous hydrogel. Other methods can create porous materials such as electro spinning Pore size can be controlled by the bead size used in the template or by the electro-spinning method employed. Pores can range from the nm size on up depending upon the application.

In one embodiment, the crosslinked cellulosic polymer can be prepared into a membrane. Such a membrane can include pores having an average dimension of about 200 nm. Also, the thin membrane can have a thickness from about 10 microns to about 100 microns or larger. Examples can be from about 20 to 80 microns, about 30 to 70 microns, about 40 to 60 microns or about 50 microns.

In one embodiment, the crosslinked cellulosic polymer can be prepared into a cell-compatible substrate for use in therapies that need cell growth, proliferation, and penetration into the biocompatible material, such as a wound which needs primary and/or secondary healing. The crosslinked cellulosic polymer can be configured as an endoprosthesis for implantation with or without cells located within the endoprosthesis. Also, the crosslinked cellulosic polymer composition can be configured as a cell culture insert that can be inserted into the well of a cell culture in vitro.

In one embodiment, the crosslinked cellulosic polymer can be configured as an article of manufacture for cell culture. The cell culture article can be conditioned to include a cell culture medium associated with the crosslinked cellulosic polymer. For example, the crosslinked cellulosic polymer can be configured into a tissue scaffold, a cell culture article, a cell culture insert, or other. The cell culture insert can have a shape configured to be received into a cell culture chamber, such as a cell culture chamber in a multi-chamber cell culture plate. The crosslinked cellulosic polymer can be porous to facilitate cell penetration, migration, and proliferation. Otherwise the composition can be non-porous to inhibit cell penetration, migration, and proliferation depending on the use.

In one embodiment, the crosslinked cellulosic polymer can be configured as a cell culture insert. Such a cell culture can be configured to fit into a cell culture chamber. The cell culture chamber can be a standalone chamber or one or many chambers in a multi-chamber plate (e.g., 96-well plate). The cell culture insert can be configured for cell migration and proliferation so that cells can migrate and proliferate through the cell culture insert. For example, the insert can be porous. Alternatively, the insert can be configured to receive cells thereon such that the cells do not penetrate or migrate into the insert which is not porous. The insert can be substantially rigid with limited flexibility, which can be represented by standard cell culture articles.

Also, one or more cells can be associated with the crosslinked cellulosic polymer. In one example, the one or more cells can include an epithelium cell. Examples of cell types can further include prokaryotic cells, eukaryotic cells, bacteria, archaea, epidermal, epidermal keratinocyte, epidermal basal cell, keratinocytes, basal cell, medullary hair shaft cell, cortical hair shaft cell, cuticular hair shaft cell, cuticular hair root sheath cell, hair matrix cell, wet stratified barrier epithelial cells, gland cells, hormone secreting cells, metabolism cells, storage cells, barrier function cells, ciliated cells, extracellular matrix secretion cells, contractile cells, blood cells, immune system cells, nervous system cells, pigment cells, germ cells, nurse cells, interstitial cells, or others as well as combinations thereof.

The crosslinked cellulosic polymer can be used in cell culture methods. Cell cultures can be grown with the composition by applying cells thereto, and then maintaining the cells with an appropriate medium. The configuration of the crosslinked cellulosic polymer can be as a cell culture insert or other cell culture article. For example, a cell culture method can include introducing a cell culture insert into a cell culture chamber, and culturing one or more cells in the cell culture chamber such that the one or more cells grow and/or proliferate. The cell culture article can allow the cells to migrate and/or proliferate on or within the cell culture insert. The cell culture method can include combining the one or more cells with the cell culture insert before, during, or after being introduced into the cell culture chamber. The cell culture method can also include introducing the one or more cells and a cell culture medium into the cell culture chamber.

The crosslinked cellulosic polymer can be configured as a tissue implant article which can be in the form of a biodegradable scaffold. The tissue implant article can be a tissue engineering scaffold with or without cells. The tissue implant article can include the crosslinked cellulosic polymer, and can include one or more cells on or in the biodegradable scaffold. Optionally, the tissue implant article can include a cell culture media in contact with the one or more cells. The one or more cells of the tissue implant can be dead or alive, and can be disperse or form a tissue.

A method of implanting cells in a subject can include obtaining a tissue implant article as described, and implanting the implant article into a subject. The implant article can be implanted with or without media in contact with the cells, and in some instances media can be removed or added before implantation.

### II. Methods for Making a Crosslinked Cellulosic Polymer

In yet another embodiment, a method for making a crosslinked cellulosic polymer is disclosed. The methods may include providing a cellulosic polymer in an aqueous solution, wherein the cellulosic polymer has reactable functional groups, activating at least a subset of the functional groups on the cellulosic polymer with a coupling agent to form a reactive intermediate, and reacting a crosslinking agent with the reactive intermediate to form either a crosslinked or a crosslinkable cellulosic polymer (e.g., having thiol groups that can be reacted to form a crosslinker). The crosslinkable cellulosic polymer can then be crosslinked (e.g., reacting the thiols to form disulfides).

The cellulosic polymer included in the aqueous solution has a molecular weight in a range from about 2000 daltons to about 2,000,000 daltons. In one aspect, the cellulosic polymer is a cellulose derivative selected from the group consisting of hydroxyethylcellulose (HEC) hydroxypropyl cellulose (HPC), carboxymethylcellulose (CMC), hydroxypropyl methylcellulose (HPMC), poly(ethylene glycol) grafted cellulose, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, and combinations thereof. In another aspect, the cellulosic polymer can be crosslinked to a starch, a pectin, an amylopectin, or a derivative thereof.

The aqueous solution may include about 0.5 wt % to about 50 wt % of the cellulosic polymer, from about 1 wt % to about 40 wt %, from about 5 wt % to about 30 wt %, from about 10 wt % to about 25 wt %, or about 20 wt %. As was explained above, the usable weight percent range for the cellulosic polymer is at least partially a function of the molecular weight of the cellulosic polymer.

The cellulosic polymer can be provided with reactable functional groups, or can be reacted with the appropriate reagents to result in the cellulosic polymer having reactable functional groups. The reactable functional groups on the cellulosic polymer may include, but are not limited to, hydroxyls, carboxlyic acids, esters, phenyl rings, benzyl groups, halogens, azides, azos, phosphates, phosphines, sulfides, sulfonyls, and the like. Reactable functional groups are well known in the chemical arts, and can be selected depending on the crosslinker as well as the crosslinking chemistry.

The reactable functional groups can be activated for substitution by crosslinking agents by adding a coupling agent to the aqueous solution. The reactable functional groups can be activated for substitution by crosslinking agents by adding at least two coupling agents to the cellulosic polymer in aqueous solution. In a specific example, the at least two coupling agent include hydroxybenzotriazole (HOBt) and a carbodiimide reagent. Suitable examples of carbodiimide reagents include, but are not limited to, N,N′-dicyclohexylcarbodiimide (DCC), N,N′-diisopropylcarbodiimide (DIC), 1-Ethyl-3-(3-dimethylaminopropyl)carbodiimide (EDC), and combinations thereof.

The coupling agents can be a well known coupling pair used in conjugation chemistry. The coupling agents can be added at a selected amount for the desired amount of crosslinking. For example, the coupling agent pair (e.g., HOBt and the carbodiimide reagent) can be added in molar excess in relation to the molar concentration of the cellulosic polymer.

After the cellulosic polymer includes the coupling agents, the crosslinking agent can then react with the coupling agents to crosslink the cellulosic polymer. Alternatively, the coupling agents can react together to form the crosslinker. The reactive ends of the crosslinking agent can be selected based on the coupling agents, or vice versa. For example, when the coupling agent includes a DCC, the crosslinking agent can include carboxylic acid groups on its ends to react with the DCC coupling agents on the monomers to be crosslinked together.

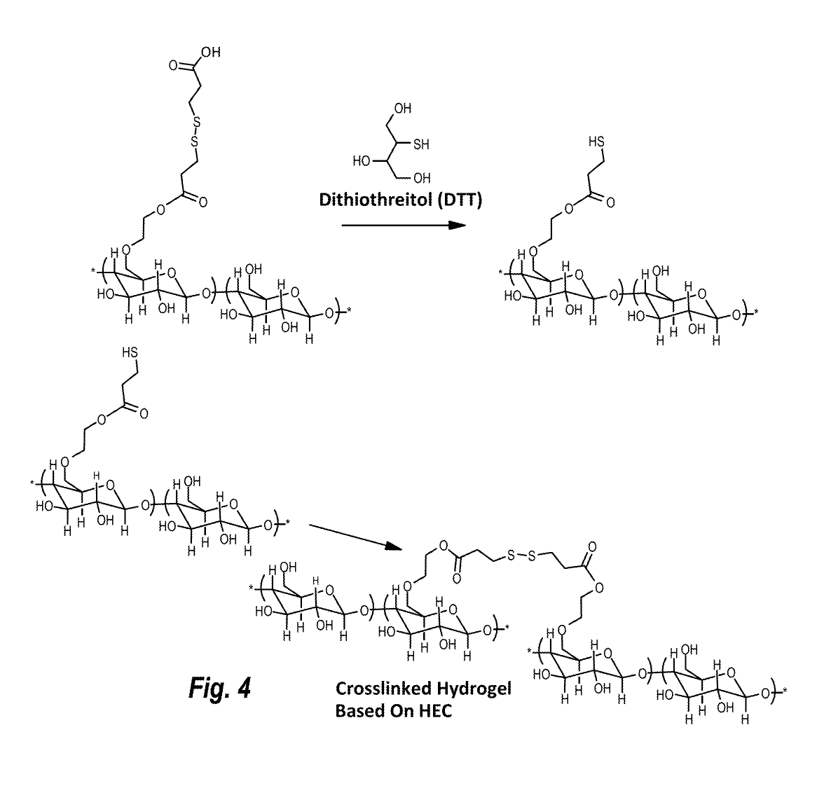

In one embodiment, the crosslinking agent may be characterized as being capable of forming a bonding interaction between a unit of a cellulosic polymer and a unit of another cellulosic polymer, where the units may be on the same polymer or different polymers. An example of such a reaction can include a linker with a reactive group on each end, where each reactive group reacts with a different reactive moiety. The reaction scheme of FIG. 4 illustrates such a reaction with a single crosslinking agent that crosslinks between two different monomers. Suitable examples of crosslinking agents useful for this type of crosslinking include, but are not limited to, dicarboxylic acids, dithio diacids, acrylics, styrenes, vinyls, urethanes, and diene/dienophile pairs, and combinations thereof. The crosslinking may also been carried out by cycloaddition reactions.

As used herein, cycloaddition reactions refer to a family of pericyclic chemical reactions, in which “two or more unsaturated molecules (or parts of the same molecule) combine with the formation of a cyclic adduct in which there is a net reduction of the bond multiplicity. Cycloaddition crosslinking can be induced by either thermal, catalytic, or photochemical means or any combination thereof.

In one embodiment, a first crosslinking agent molecule may be capable of forming a bonding interaction with a first cellulosic polymer molecule and a second crosslinking agent molecule may be capable of forming a bonding interaction with a second cellulosic polymer molecule. The crosslinking agent may be characterized as being capable of reacting together and/or with a third crosslinking agent to form a crosslinker that crosslinks two or more cellulosic monomers. Suitable examples of crosslinking agents include, but are not limited to, thiols, acrylics, styrenes, vinyls, urethanes, and diene/dienophile pairs, a Diels-Alder pair (i.e., a diene and a dienophile), and combinations thereof.

In one aspect, the method may include crosslinking about 0.01% to about 20% of the functional groups on the cellulosic polymer with a crosslinker. In another aspect, about 0.1%, about 0.5%, about 1%, about 2%, about 3%, about 4%, about 5%, about 6%, about 7%, about 8%, about 9%, about 10%, about 11%, about 12% about 13%, about 14%, about 15%, about 16%, about 17%, about 18%, about 19%, or about 20% of the functional groups on the cellulosic polymer can be linked to a crosslinker. Based on the foregoing discussion, one will appreciate that the proportion of functional groups that are linked with the crosslinker is at least partially a function of the concentration of coupling agent and/or crosslinking agent that is added to the cellulosic polymer in the crosslinking reaction.

In one aspect, the method may further include treating the crosslinked cellulosic polymer to at least partially reverse the crosslinking For example, if the crosslinking agent forms a crosslinker with a disulfide linkage, the crosslinking can be reversed (i.e., the disulfide bond can be broken) by adding dithiothreitol or a similar reducing agent. Reversing the crosslinking can form a crosslinkable cellulosic polymer.

In another aspect, the method may further include treating the crosslinkable cellulosic polymer to reform the crosslinker so as to crosslink the cellulosic polymer. In the case of the thiols crosslinking agents and the disulfide crosslinkers, the crosslink can be reformed by adding hydrogen peroxide or another oxidizing reagent to the crosslinkable cellulosic polymer to form a crosslinked cellulosic polymer. Additional crosslinkers (or crosslinking agents) can include polyester crosslinkers or crosslinkers that have ester moieties, poly(ethylene glycol) crosslinkers, Diels-Alder crosslinkers that are reversible, and disulfide crosslinkers as well as the crosslinking agents that form the crosslinkers.

In one aspect, the method can further include dialyzing the crosslinked cellulosic polymer to remove unreacted coupling agent and crosslinking agent. For example, the crosslinked cellulosic polymer can be placed into a dialyzing chamber with solvent (e.g., water) and the unreacted coupling agents and crosslinking agents can diffuse out from the dialyzing chamber.

In another aspect, the method can further include drying the crosslinked or crosslinkable cellulosic polymer. Drying can include one or more of evaporating the aqueous solution or precipitating the cellulosic polymer out of the aqueous solution. For example, the aqueous solution can be evaporated under heat and/or under vacuum. The cellulosic polymer can be precipitated out of solution by the addition of non-polar solvents to the aqueous solution that make the cellulosic polymer precipitate out of solution.

In another aspect, the crosslinking can be performed in the presence of a porogen. The porogen can then be removed from the crosslinked cellulosic polymer. The porogen can be an inorganic salt like sodium chloride, crystals of saccharose, gelatin spheres or paraffin spheres. The size of the porogen particles can affect the size of the pores, while the polymer to porogen ratio is directly correlated to the amount of porosity of the final structure. After the crosslinked polymer has been formed, the reaction solvent is allowed to fully evaporate, then the crosslinked polymer is immersed in a bath of a liquid suitable for dissolving the porogen. Water can be used to dissolve porogens of sodium chloride, saccharose and gelatin. An aliphatic solvent like hexane can be used for paraffin. Once the porogen has been fully dissolved a porous structure is obtained.

In one aspect, the pores can be formed without a porogen. First, structures made of the crosslinked cellulosic polymer are prepared. The structures are then placed in a chamber where are exposed to high pressure CO₂ for several days. The pressure inside the chamber is gradually restored to atmospheric levels. During this procedure the pores are formed by the carbon dioxide molecules that leave the crosslinked cellulosic polymer, resulting in a sponge like structure.

Formulas 1 and 2 above provide schematic representations of a cellulose polymer (Formula 1) and a cellulosic polymer (Formula 2) having derivatized cellulose. The cellulosic polymer can include cellulosic monomer units that are linked by glycosidic bonds. The cellulosic monomer units can be any cellulose or derivative thereof. Cellulosic polymers are linear. Cellulosic polymers 100 can be homologous, or heterogeneous by containing one or more monomers that have been functionalized or otherwise substituted.

As illustrated in Formula 2, each monomer can include one to three R groups, where the R groups can be the same or each can be different. And while three R groups are shown for the purpose of illustration, each monomer can have fewer R groups depending on the particular cellulosic monomer. In most naturally occurring cellulosic polymers, R is H.

Cellulosic polymers can also be derivatized under conditions that are usually strongly basic to produce a number of cellulosic polymer derivatives. Suitable examples of R groups that can be attached to the cellulosic monomers can include, but are not limited to, straight or branched substituted or unsubstituted C1-C20 alkane, straight or branched substituted or unsubstituted C1-C20 alkene, straight or branched substituted or unsubstituted C1-C20 alkyne, straight or branched substituted or unsubstituted C1-C20 carboxlyic acid, straight or branched substituted or unsubstituted C1-C20 ester, phenyl, benzyl, halogen, straight or branched substituted or unsubstituted alkoxy, primary amine, secondary amine, tertiary amine, azide, azo, phosphate, phosphine, sulfide, sulfonyl, substituted or unsubstituted cycloalkyl, substituted or unsubstituted aryl, branched or unbranched or cyclic substituted or unsubstituted arylalkyl, or combinations thereof. Suitable R groups can also include dye molecules such as coumarins and coumarin derivatives, rhodamine and rhodamine derivatives, fluorescein and fluorescein derivatives (e.g., fluorescein isothiocyanate), congo red, methyl red, and the like.

With regard to Formulas 1 and 2, “n” can range from about 10 and about 30,000, about 50 to about 20,000, about 100 to about 10,000, about 500 to about 5,000, or any range therebetween. A particular example can be around 6,000.

Because it is essentially insoluble in water, cellulose can be functionalized or derivatized to be usable in the present disclosure. There are, however, a number of common cellulose derivatives that are soluble. One such derivative, hydroxyethylcellulose (HEC), is illustrated in Formula 2 with one or more R groups being CH₂CH₂OH. Each monomer can include 1, 2, or 3 R groups that can be replaced by CH₂CH₂OH. A typical HEC polymer can contain a blend of singly, doubly, and triply substituted monomer units. Other common cellulose derivatives include, but are not limited to, hydroxypropyl cellulose (HPC), carboxymethylcellulose (CMC), hydroxypropyl methylcellulose (HPMC) where R can be CH₂CH(OH)CH₃, CH₂CO₂H, and CH₂CH(OH)CH₃ and CH₃, respectively.

Cellulosic polymers based on cellulose derivatives may have a number of advantages. For example, low to zero toxicity, the products of their degradation products are sugars (mainly glucose), there is no known inflammatory response to the degradation products of cellulose derivatives, their degradation rate is tunable by crosslink density, and their degradation rate can be tuned to specific applications. Moreover, cellulose derivatives may be used in either hydrogels or in solid films, cellulose derivatives are cost effective (e.g., HEC cost about $0.20/kg), the materials are readily available through high volume sources, food and pharmaceutical grade materials are available, and they can be co-polymerized with a variety of monomers.

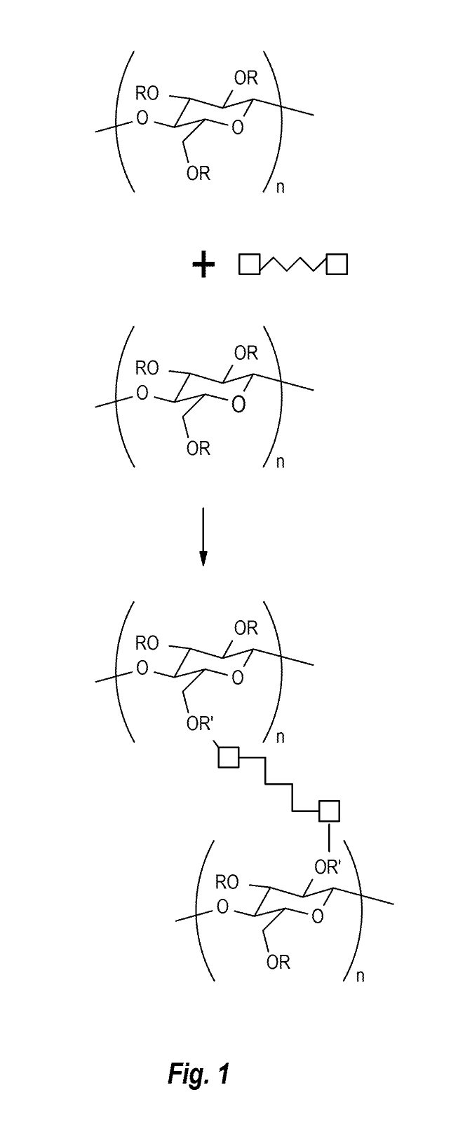

Referring now to FIG. 1, a schematic of an illustrative embodiment of a monomer of a cellulosic polymer that is crosslinked to another monomer of a cellulosic polymer is depicted. In the depicted embodiment, one of the R groups on each of the cellulosic polymers is reacted with a crosslinking agent (e.g., where the squares represent reactive groups on the crosslinking agent) so that the R groups are converted to R′ groups that are linked to the crosslinker. One will appreciate, however, that in other embodiments the crosslinking agent can be attached to an R group as well as replacing an R group.

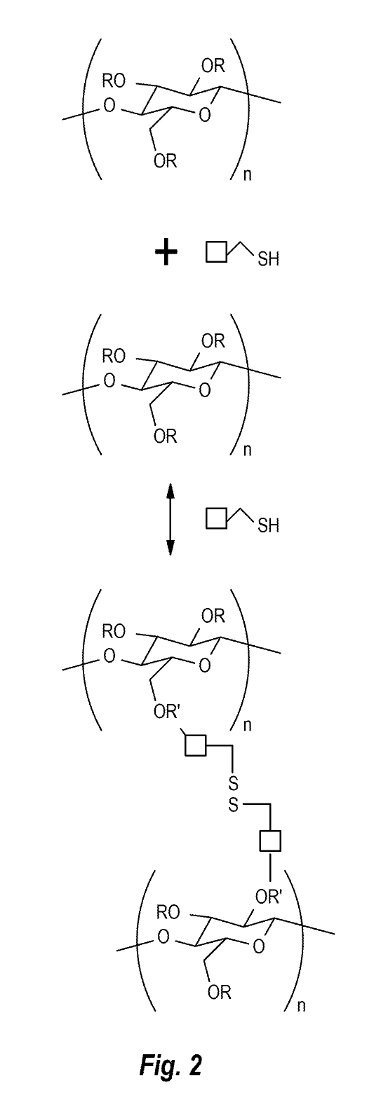

Referring now to FIG. 2, a schematic of an illustrative embodiment of a reversibly crosslinkable cellulosic polymer is depicted. As shown, a first crosslinkable cellulosic polymer is linked to a reversibly crosslinkable crosslinker at a reactive group (e.g., shown as the square) that is opposite of the terminal thiol group. The second crosslinkable cellulosic polymer is also linked to a reversibly crosslinkable crosslinker having a terminal thiol group. The two thiol groups of the two different reversibly crosslinkable crosslinkers can then couple together through a disulfide bond. However, the terminal thiol groups of the linkers can be replaced by other reactive moieties that react together to crosslink the cellulosic polymers. For example, the cellulosic polymers can be crosslinked to form a crosslinked cellulosic polymer by adding a reagent, catalysts, irradiation (e.g., UV radiation), or the like that will stimulate the formation of crosslinked cellulosic polymer. When a disulfide bond links the crosslinkers together, the crosslinked cellulosic polymer may also be reversed to the uncrosslinked version by the addition of a reducing agent or a similar reagent that can sever the disulfide bond.

A number of illustrative Examples will now be referred to. The Examples are intended solely to clarify the present disclosure and are not intended to limit the present disclosure in any way.

### EXAMPLES

### Example 1

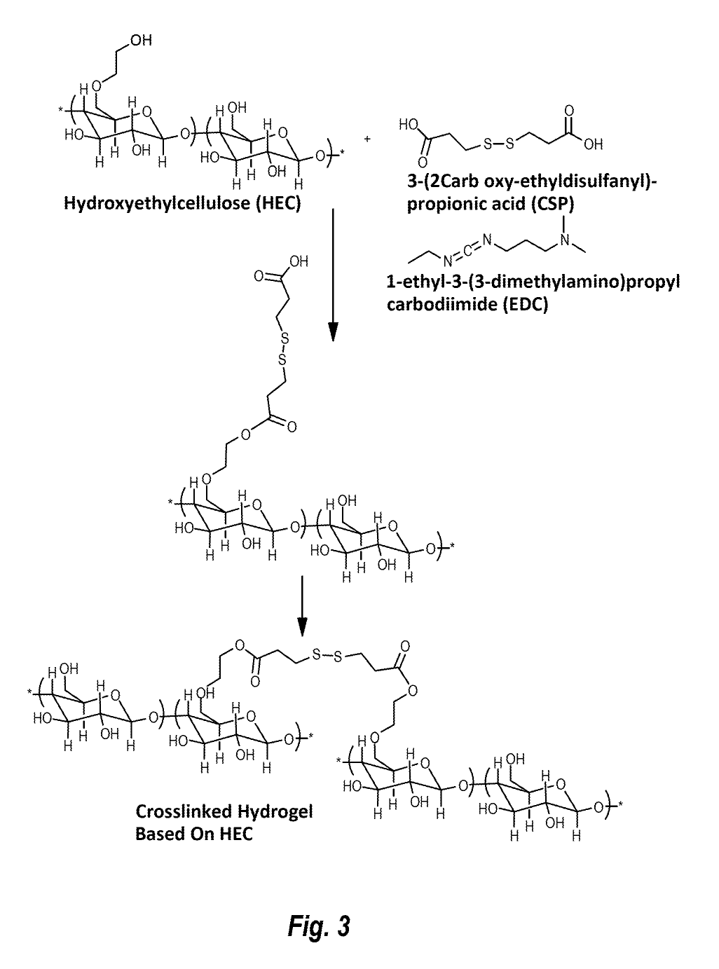

### Synthesis of 3-(2-Carboxy-ethyldisulfanyl)-propionic acid (CSP) crosslinked HEC hydrogel (FIG. 3)

Five grams of Natrosol LR 250 Pharm is dissolved in 100 mL of phosphate buffer saline (PBS) solution (pH 7.0) and mixed well until no aggregation is observed. Natrosol 250LR Pharm, a pharmaceutical grade HEC, is a good choice since it is relatively low in molecular weight (90,000) allowing for higher concentrations of the polymer to be used without creating unworkable viscosity which would make stirring of the solutions difficult. A 5% solution of Natrosol 250LR has a Brookfield viscosity of 75-150. The lower the molecular weight of HEC used the higher the solids content of the hydrogels that can be achieved.

Six molar excess amounts of 1-ethyl-3-(3-dimethylamino)propylcarbodiimide (EDC) (38 mmol) and 1-hydroxybenzotriazole (HOBt) (200 mmol) are then added and stirred for 2 h, and then CSP (37.5 mmol) is added dropwise and mixed. At this time the solution can be molded into the desired shape. The reaction mixture is heated to 60° C. for 24 hours to produce the crosslinked hydrogel. The Hydrogel is then dialyzed for 24 h to remove unreacted CSP, EDC and HOBt coupling agents.

### Example 2

### Synthesis of Functionalized, Crosslinkable HEC (FIG. 4)

Two grams of Natrosol LR 250 Pharm are dissolved in 125 ml of PBS solution (pH 7.0) and mixed well until no aggregation is observed. Three molar excess amounts of 1-ethyl-3-(3-dimethylamino)propylcarbodiimide (EDC) (18 mmol) and 1-hydroxybenzotriazole (HOBt) (95 mmol) are added and stirred for 2 h, and then CSP (18 mmol) is added dropwise and mixed overnight. CSP conjugated HEC was treated with five molar excess of dithiothreitol (DTT) and stirred for 24 h to cleave the disulfide bond in CPS. Thiol functionalized HEC (HEC-SH) was dialyzed against deionized water at pH 3.0 for 2 days. The final product is lyophilized for 3 days and stored at −20° C. until use. Percent thiol modification can be determined by the Elman's assay using L-cysteine as a standard.

To form a crosslinked hydrogel of HEC-SH two grams of HEC-SH is dissolved in 10 ml of PBS solution at pH 7.0 until no aggregation is observed. A viscous clear solution of HEC-SH is the result. Crosslinking occurs via the reaction 2 RSH→RS—SR+2 H++2 e−. A small amount of hydrogen peroxide (0.1-1% HEC wt.) can be added to catalyze the reaction.

### Example 3

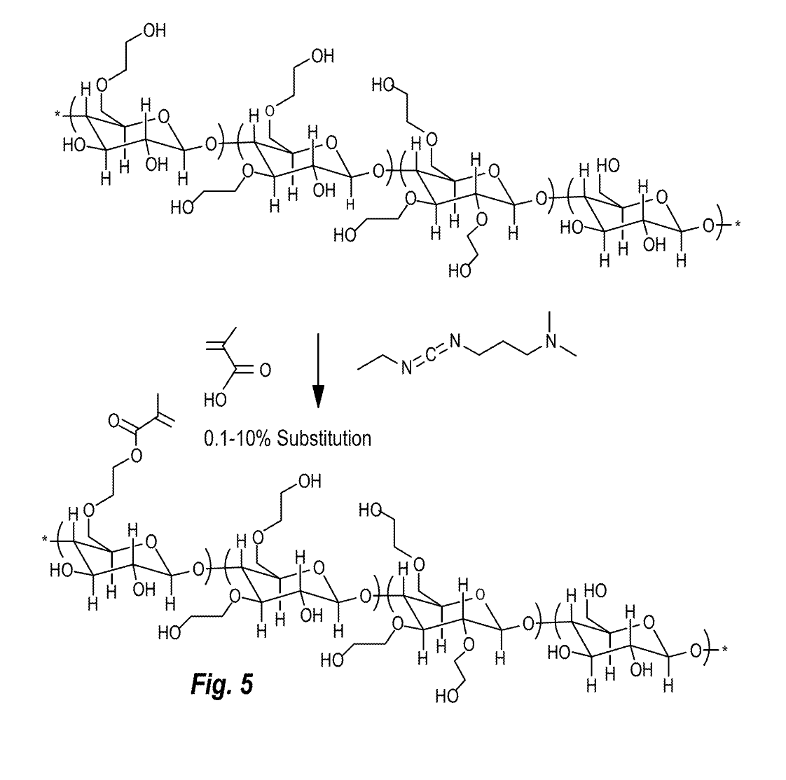

### Synthesis of Acrylic Functionalized HEC (FIG. 5)

To a 500 mL flask is added 100 mL of de-ionized water and a magnetic stir bar. With rapid stirring 2.0 g of HEC (Natrosol LR250Pharm) is added to the water and allowed to dissolve. The resulting solution of Natrosol is used to make MMA-HEC methacrylic acid and other components used in 0.1 to 20 times weight of HEC.

1-Ethyl-3-(3-dimethylamino)propylcarbodiimide (EDC) and 1-hydroxybenzotriazole (HOBt) (3× the EDC mol used) are added and stirred for 2 h, and then methacrylic acid (equal mol to EDC) is added dropwise and mixed overnight. The newly synthesized MMA-HEC was precipitated by the addition of 100 mL THF, pouring into a 1 L beaker, and then adding ˜800 mL of acetone. The MMA-HEC was isolated as a white precipitate. The crude GM-HEC was purified by repeated precipitation; a total of three times by redissolving into 50 mL of water then precipitating by 100 mL THF followed by 800 mL acetone. The residue was washed with acetone to remove water and then placed in a vacuum oven at 60° C. overnight.

### Example 4

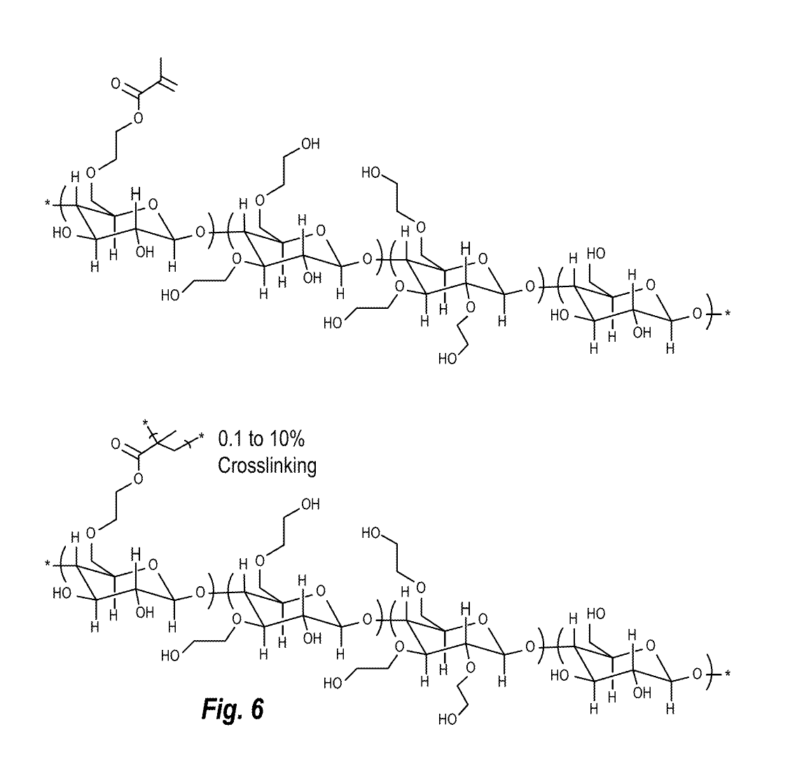

### Synthesis of Crosslinked GM-HEC Hydrogel (FIG. 6)

MMA-HEC (Example 3) is used to create hydrogels. MMA-HEC is dissolved into water over a period of two hours with stirring at 40° C. One drop (0.030 g) of Darocure 1173 is added and well mixed into the solution, and the solution purged of oxygen. The GM-HEC is polymerized by exposure to UV radiation (300-400 nm) for 3 minutes. The MMA-HEC can also be polymerized with co-monomers such as vinyl ether, 2-hydroxyethyl methacrylate (HEMA), n-vinyl pyrrolidone (NVP), polyethyleneglycol dimethacrylate (PEGDMA), and the like.

Crosslinked cellulosic polymers can be used as the matrix for tissue engineering in cell growth scaffolds. There are a variety of designs and architectural methods to scaffolds. The cellulose polymers described here are the materials of which the scaffolds are constructed.

### Example 5

### Electro-Spinning

Fibrous mats of HEC can be formed by means simultaneous crosslinking and electro-spinning. In this situation an electro-spinning apparatus is equipped with a high-voltage statitron. HEC and crosslinkers are dissolved in water to prepare a 10% solution, and added to a 2 mL glass syringe, which is attached with a clinically shaped metal capillary. The flow is controlled by a precision pump to maintain a steady flow of 0.5 mL/hr from the capillary outlet. The electro-spun fibers are deposited on a rotating frame cylinder collector consisting of metal struts. When using the frame consisting of metal struts as the collector, the electrostatic forces drive the fibers to move towards the metal struts. Fibers of higher density are deposited on the metal struts while fibers of lesser density are deposited between the struts. The rotating speed of the cylinder collector is controlled by a stepping motor. The deposition time can be optimized to obtain fibrous mats with thicknesses of 250-300 μm. All the non-woven fibrous mats were vacuum-dried at room temperature for 3 days to completely remove any solvent residue prior to further characterization.

### Example 6

### Constructs with Spherical Pores

Poly(methyl methacrylate) (PMMA) microspheres with diameter 90±10 μm are manufactured as porogen templates by introducing them between two plates whose distance can be controlled by adjusting the step of a coupled screw and heated at 180° C. for 30 min to obtain the first template. This template shows the highest porosity attainable with typical compaction values of 60-65% for random monosized spherical particles. To obtain scaffolds with controlled porosity, the thickness of the obtained disk was first measured; then the disk was replaced in the mould and compressed at 180° C. for half an hour. The degree of compression was quantified by measuring the thickness diminution.

After cooling the template at room temperature, a 15% HEC solution in water is introduced in the empty space between the PMMA spheres. The polymerization is carried out by heating the HEC solution in the template to 60° C. for up to 24 hours.

After polymerization, the porogen template was removed by Soxhlet extraction with acetone. The porous sample is then extracted with ethanol to extract low molecular weight substances. Samples are then dried in vacuum to constant weight before characterization. The crosslinked porous samples can be re-swelled for use with water and/or aqueous buffer.

### Example 7

### Scaffolds Formed from Emulsion

HEC solutions are prepared by dissolution in PBS with crosslinker. One to ten percent 50:50 PLGA solutions of various MWs are prepared by dissolving in chloroform. 1,2-dimyristoyl-sn-glycero-3-phosphocholine (DMPC) solution is prepared in chloroform. One milliliter of chloroform solutions are mixed with 10-40 μA DMPC solution and then the mixture is added to 3 mL of the HEC solution. After addition, the samples are capped and mixed by either placing on a mini-vortexer at 3200 rpm for 3-4 min, or by sonication for 90 s at 50% amplitude by a 500 Sonic Dismembrator. These samples are used for scaffold formation.

Portions of the blended emulsions are poured into flat-bottomed 15 mm diameter Nalgene tubes. Freezing is accomplished by placing these tubes in a commercial freezer, on dry ice, or on liquid nitrogen with respective temperatures of −20, −78, and −196° C. After the samples frozen at −20 and −78° C. equilibrated at their respective temperatures, they were subsequently placed in liquid nitrogen prior to lyophilization. All samples were lyophilized until dry.

The porous sample is then extracted with ethanol to extract low molecular weight substances. Samples are then dried in vacuum to constant weight before characterization. The crosslinked porous samples can be re-swelled for use with water and/or aqueous buffer.

### Example 8

### Diels Alder Crosslinking

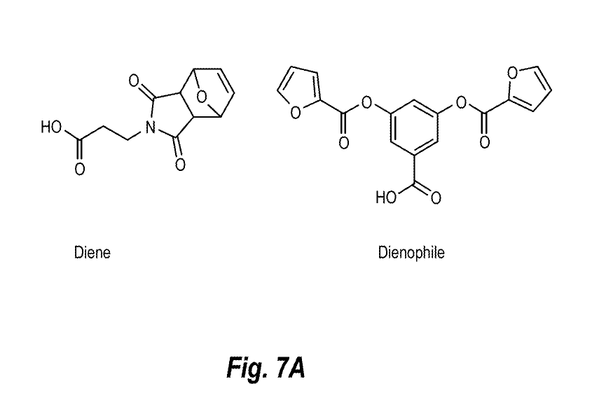

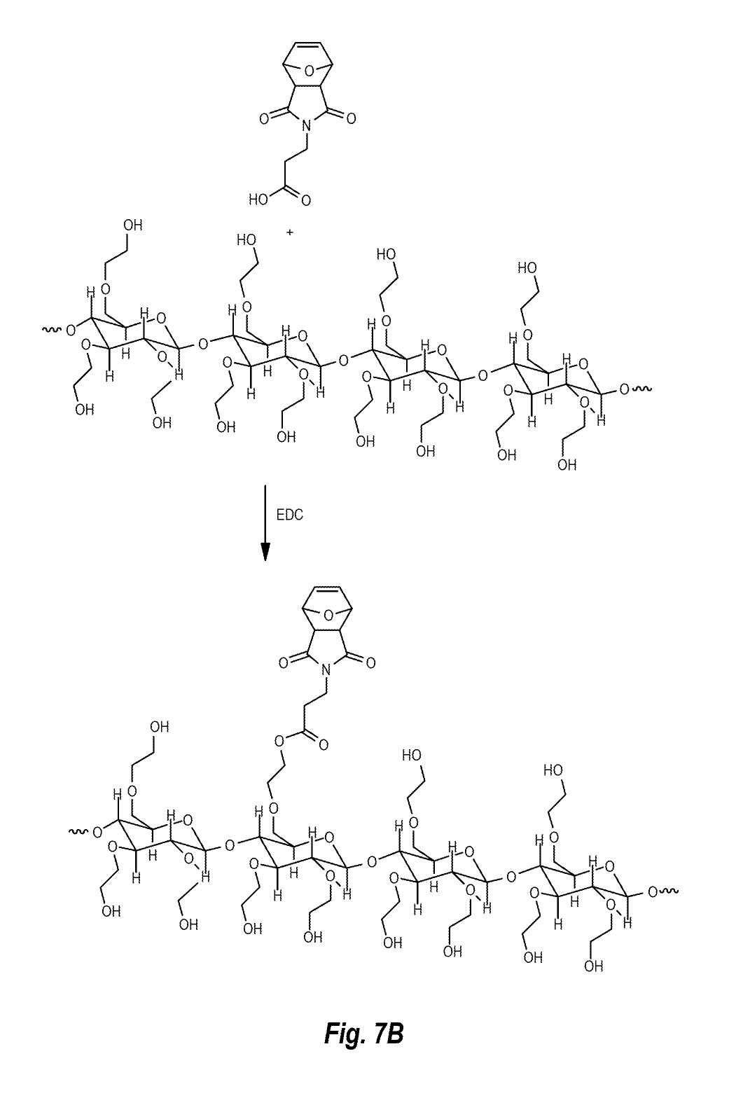

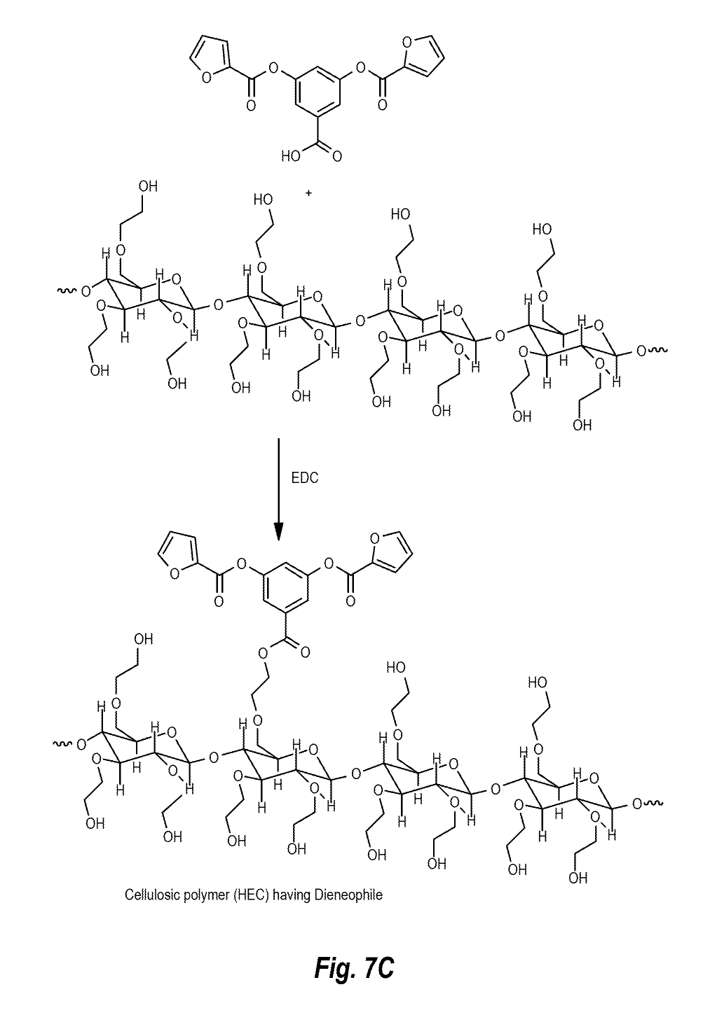

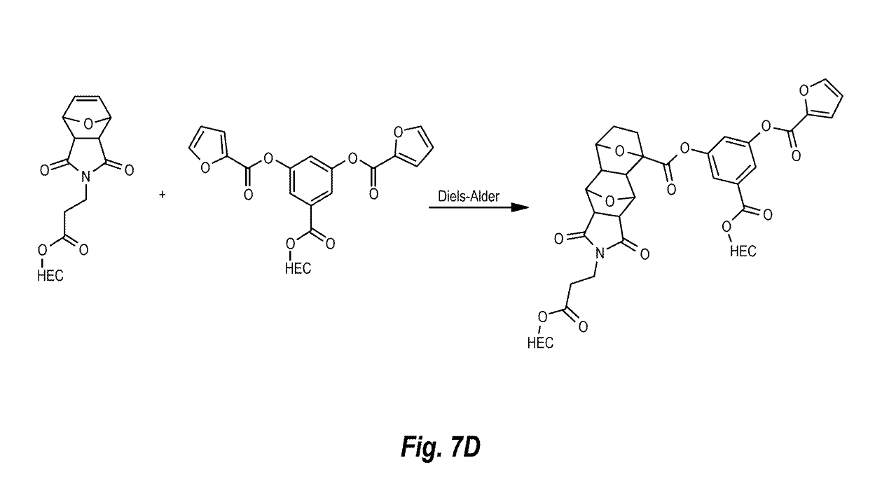

FIGS. 7A-7D provide embodiments of Diels Alder dienophile and diene and corresponding reactions with the cellulosic polymer (HEC) as well as crosslinking reaction between the dienophile and diene to crosslink the HEC polymers.

The present disclosure is not to be limited in terms of the particular examples described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular examples only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-129. (canceled)

130. A cell growth scaffold adapted for implantation in a body, the cell growth scaffold comprising:
one or more cellulosic polymers;
an anesthetic compound; and
one or more crosslinkers crosslinking the one or more cellulosic polymers to form a crosslinked cellulosic polymer, wherein the crosslinked cellulosic polymer is adapted to break down in the body over time after implantation, yielding breakdown products that are non-toxic and non-irritating to the body.

131. The cell growth scaffold of claim 130, wherein the breakdown products include at least one of oligosaccharides, oligosaccharide derivatives, or sugar monomer units.

132. The cell growth scaffold of claim 130, wherein the crosslinked cellulosic polymer has a molecular weight of about 2000 daltons to about 500,000 daltons.

133. The cell growth scaffold of claim 130, wherein the one or more cellulosic polymers includes a cellulose derivative.

134. The cell growth scaffold of claim 130, wherein the one or more cellulosic polymers are selected from the group consisting of hydroxyethylcellulose (HEC) hydroxypropyl cellulose (HPC), carboxymethylcellulose (CMC), hydroxypropyl methylcellulose (HPMC), poly(ethylene glycol) grafted cellulose, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, and combinations thereof.

135. The cell growth scaffold of claim 130, wherein the one or more crosslinkers are the product of a reaction between the one or more cellulosic polymers and a crosslinking agent selected from the group consisting of a dithio diacid, a dicarboxylic acid, an acrylic moiety, a diazide, a styrene, a vinyl carboxylic acid, a urethane, a vinyl acetate, a vinyl ether, a Diels-Alder reagent, disulfides, photopolymerizable moiety, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, derivatives thereof, and combinations thereof.

136. The cell growth scaffold of claim 130, wherein the one or more crosslinkers are at least one of photoreactive or thermoreactive

137. The cell growth scaffold of claim 130, further comprising a dye linked to the one or more cellulosic polymers.

138. The cell growth scaffold of claim 130, wherein the cell growth scaffold includes an aqueous medium.

139. The cell growth scaffold of claim 138, wherein the aqueous medium includes at least one of a buffering agent or a cell growth factor.

140. A method for growing a tissue in a body, the method comprising:
providing a cell growth scaffold that includes one or more cellulosic polymers and one or more crosslinkers crosslinking the one or more cellulosic polymers to form a crosslinked cellulosic polymer;
implanting the cell growth scaffold into a body of a subject where one or more cells can at least one of migrate into the cell growth scaffold or proliferate on or within the cell growth scaffold,
wherein the crosslinked cellulosic polymer is adapted to break down in the body over time after implantation, yielding breakdown products that are non-toxic and non-irritating to the body.

141. The method of claim 140, wherein the breakdown products include at least one of oligosaccharides, oligosaccharide derivatives, or sugar monomer units.

142. The method of claim 140, wherein the cell growth scaffold is substantially free of cells prior to implanting the cell growth scaffold into the body.

143. The method of claim 140, further comprising at least one of implanting one or more cells onto or into the cell growth scaffold prior to implanting the cell growth scaffold into the body.

144. The method of claim 140, wherein the cell growth scaffold is provided in a dehydrated state.

145. The method of claim 144, further comprising rehydrating the cell growth scaffold with an aqueous medium prior to implanting the cell growth scaffold into the body.

146. The method of claim 145, wherein the aqueous medium includes at least one of a buffering agent or a cell growth factor.

147. The method of claim 144, further comprising implanting the cell growth scaffold into the body in the dehydrated state.

148. The method of claim 140, wherein the one or more cellulosic polymers are selected from the group consisting of hydroxyethylcellulose (HEC) hydroxypropyl cellulose (HPC), carboxymethylcellulose (CMC), hydroxypropyl methylcellulose (HPMC), poly(ethylene glycol) grafted cellulose, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, and combinations thereof.

149. The method of claim 140, wherein the one or more crosslinkers are the product of a reaction between the one or more cellulosic polymers and a crosslinking agent selected from the group consisting of a dithio diacid, a dicarboxylic acid, an acrylic moiety, a diazide, a styrene, a vinyl carboxylic acid, a urethane, a vinyl acetate, a vinyl ether, a Diels-Alder reagent, disulfides, photopolymerizable moiety, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, derivatives thereof, and combinations thereof.

150. A method for growing a tissue in a body, the method comprising:
providing a cell growth scaffold that includes:
one or more cellulosic polymers having crosslinkable functional groups; and
one or more crosslinkers capable of reacting with the crosslinkable functional groups to crosslink the one or more cellulosic polymers to form a crosslinked cellulosic polymer; and
implanting the cell growth scaffold into a body of a subject where one or more cells can at least one of migrate into the cell growth scaffold or proliferate on or within the cell growth scaffold,
wherein the crosslinked cellulosic polymer is adapted to break down in the body over time after implantation, yielding breakdown products that are non-toxic and non-irritating to the body.

151. The method of claim 150, wherein the crosslinked cellulosic polymer has a viscosity selected to allow the cell growth scaffold to hold a selected shape after implantation into the body.

152. The method of claim 151, wherein the wherein the viscosity of the crosslinked cellulosic polymer is stable under shear.

153. The method of claim 150, wherein about 0.01% to about 20% of the crosslinkable functional groups of the one or more cellulosic polymers are linked to a crosslinking agent.

154. The method of claim 150, wherein about 0.1% to about 15% of the functional groups of the one or more cellulosic polymers are linked to a crosslinking agent.

155. The method of claim 150, wherein about 0.1% to about 10% of the functional groups of the one or more cellulosic polymers are linked to a crosslinking agent.

156. The method of claim 150, wherein the crosslinked cellulosic polymer includes at least one of an aqueous buffering agent or a cell growth factor.

157. The method of claim 150, wherein the cellulosic polymer is selected from the group consisting of hydroxyethylcellulose (HEC) hydroxypropyl cellulose (HPC), carboxymethylcellulose (CMC), hydroxypropyl methylcellulose (HPMC), poly(ethylene glycol) grafted cellulose, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, and combinations thereof.

158. The method of claim 150, wherein the crosslinker is the product of a reaction between the one or more cellulosic polymers and a crosslinking agent selected from the group consisting of a dithio diacid, a dicarboxylic acid, an acrylic moiety, a diazide, a styrene, a vinyl carboxylic acid, a urethane, a vinyl acetate, a vinyl ether, a Diels-Alder reagent, disulfides, photopolymerizable moiety, acrylic acid grafted cellulose, hydroxymethyl methacrylate grafted cellulose, poly(vinyl alcohol) grafted cellulose, poly(vinyl amine) grafted cellulose, acrylamide grafted cellulose, polyallylamine-grafted cellulose, cellulose containing gluconic acid, derivatives thereof, and combinations thereof.
